(19) United States
(12) Patent Application Publication
(10) Pub. No.: US 2024/0033911 A1
(43) Pub. Date: Feb. 1, 2024

(54) CONTROL SYSTEM AND CONTROL METHOD

(71) Applicant: SONY GROUP CORPORATION, TOKYO (JP)

(72) Inventor: SHUNSUKE YAJIMA, TOKYO (JP)

(21) Appl. No.: 18/258,415

(22) PCT Filed: Nov. 24, 2021
(86) PCT No.: PCT/JP2021/042873

§ 371 Date: Jun. 20, 2023

(30) Foreign Application Priority Data
Dec. 28, 2020 (JP) 2020-219253

(51) Int. Cl. B25J 9/16; B25J 3/04; B25J 13/08; A61B 34/37

(52) U.S. Cl. CPC B25J 9/1633; B25J 3/04; B25J 9/1689; B25J 13/085; A61B 34/37

(57) ABSTRACT

A control system (1) includes: an estimation unit (16) that estimates future state information of a slave robot (21) at a time after a predetermined period from a time corresponding to state information of a master robot (11) and the slave robot (21), the state information being acquired by a master device (10) among the master device (10) and a slave device (20), the master device (10) including the master robot (11) while the slave device (20) including the slave robot (21), the estimation of the future state information being performed by using the state information of the master robot (11) and the slave robot (21) and using a trained model (NN); and a controller (12, 13, 22, 23) that controls the master robot (11) and the slave robot (21) based on an estimation result obtained by the estimation unit (16) so as to achieve a correspondence between a position and an external force of the master robot (11) and a position and an external force of the slave robot (21), respectively. The trained model (NN) works such that, after having input of the state information, the trained model (NN) outputs the future state information.

### FIELD

The present disclosure relates to a control system and a control method.

### BACKGROUND

Various proposals have been made regarding a master-slave control system (refer to Patent Literature 1, for example).

### CITATION LIST

### Patent Literature

Patent Literature 1: WO 2019/012812 A

### SUMMARY

### Technical Problem

In bilateral control in which the position and force are made to correspond to each other between a master and a slave, overcoming a communication delay between the master and the slave leads to improvement of operability.

One aspect of the present disclosure is to provide a control system and a control method capable of improving operability of bilateral control.

### Solution to Problem

A control system according to one aspect of the present disclosure includes: an estimation unit that estimates future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and a controller that controls the master robot and the slave robot based on an estimation result obtained by the estimation unit so as to achieve a correspondence between a position and an external force of the master robot and a position and an external force of the slave robot, respectively, wherein the trained model works such that, after having input of the state information, the trained model outputs the future state information.

A control method according to one aspect of the present disclosure includes: estimating, by a control system, future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and controlling, by the control system, the master robot and the slave robot based on an estimation result so as to achieve a correspondence between a position and an external force of the master robot and a position and an external force of the slave robot, respectively.

### BRIEF DESCRIPTION OF DRAWINGS

FIG. 1 is a diagram illustrating an example of a schematic configuration of a control system according to an embodiment.

FIG. 2 is a diagram illustrating an example of a configuration of a master device.

FIG. 3 is a diagram illustrating an example of a configuration of a slave device.

FIG. 4 is a diagram illustrating an example of a schematic configuration of a trained model.

FIG. 5 is a diagram schematically illustrating an example of acquisition of training data.

FIG. 6 is a diagram schematically illustrating an example of training data.

FIG. 7 is a diagram schematically illustrating an example of bilateral control.

FIG. 8 is a flowchart illustrating an example of processing (control method) executed in the control system.

FIG. 9 is a diagram illustrating an example of a hardware configuration of the device.

### DESCRIPTION OF EMBODIMENTS

Embodiments of the present disclosure will be described below in detail with reference to the drawings. Note that, in each of the following embodiments, the same elements are denoted by the same reference symbols, and a repetitive description thereof will be omitted.

The present disclosure will be described in the following order.
1. Embodiments
2. Modification
3. Hardware configuration example
4. Effects

### 1. Embodiments

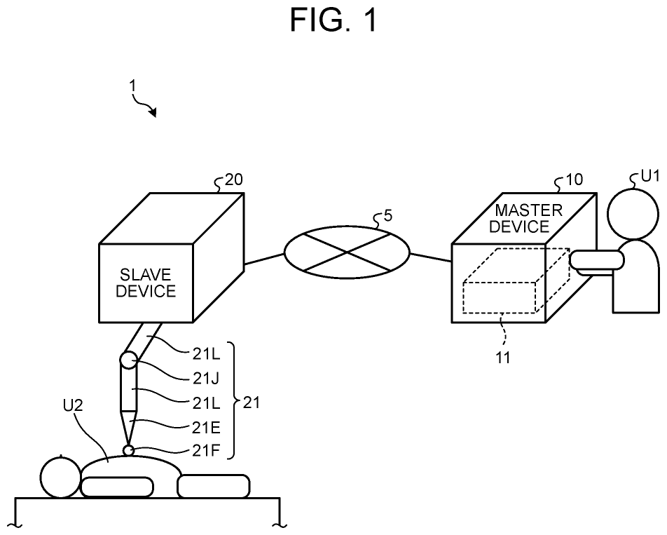

FIG. 1 is a diagram illustrating an example of a schematic configuration of a control system according to an embodiment. A control system 1 is a control system using a master-slave method, and includes a master device 10 and a slave device 20. These devices are communicably connected via a network 5. Information (such as data) used or acquired by individual devices is transmitted and received between the devices as necessary. An example of the network 5 is the Internet. The network 5 may include a wired network, a wireless network, and the like.

In this example, the control system 1 is used for surgery. A practitioner (doctor or the like) who performs the surgery is referred to as a user U1 in the drawing. A patient undergoing the surgery is referred to as a user U2 in the drawing.

By operating the master device 10, the user U1 remotely operates the slave device 20 to perform surgery on the user U2. Specifically, the master device 10 includes a master robot 11 operated by the user U1. The slave device 20 includes a slave robot 21 remotely controlled by the user U1. For convenience, the slave robot 21 and the master robot 11 will be described in this order.

In this example, the slave robot 21 includes an arm. The slave robot 21 in this example includes a link 21L, a joint 21J, and a distal end 21E. The links 21L provided adjacent to each other are pivotally connected via the joint 21J that rotates by motor drive. The distal end 21E is a portion that is located closest to the user U2 and can come in contact with the user U2. The slave robot 21 has various states depending on conditions such as the rotation angle of the joint 21J, for example. A force sensor 21F is provided at the distal end 21E. The force sensor 21F detects a reaction force (external force) acting on the distal end 21E. Note that the slave robot 21 may include more numbers of links 21L and joints 21J, and more numbers of distal ends 21E and the force sensors 21F, than the numbers in the diagram. The following description regarding the link, the joint, and the distal end may be appropriately read as each link, each joint, and each distal end.

The master robot 11 has a configuration suitable for the user U1 to remotely operate the slave robot 21. Information regarding the operation (user's operation) of the master robot 11 by the user U1 is transmitted as control information from the master device 10 to the slave device 20. The master robot 11 has a configuration corresponding to the slave robot 21 so as to transmit the state of the slave robot 21 to the user U1. For example, the master robot 11 includes portions corresponding to the respective portions of the slave robot 21 so as to have states corresponding to the states of the respective portions (link 21L, joint 21J, distal end 21E, and the like) of the slave robot 21. Hereinafter, a portion in the master robot 11 corresponding to the joint 21J of the slave robot 21 is referred to as a “joint of the master robot 11”. A portion in the master robot 11 corresponding to the distal end 21E of the slave robot 21 is referred to as a “distal end of the master robot 11”.

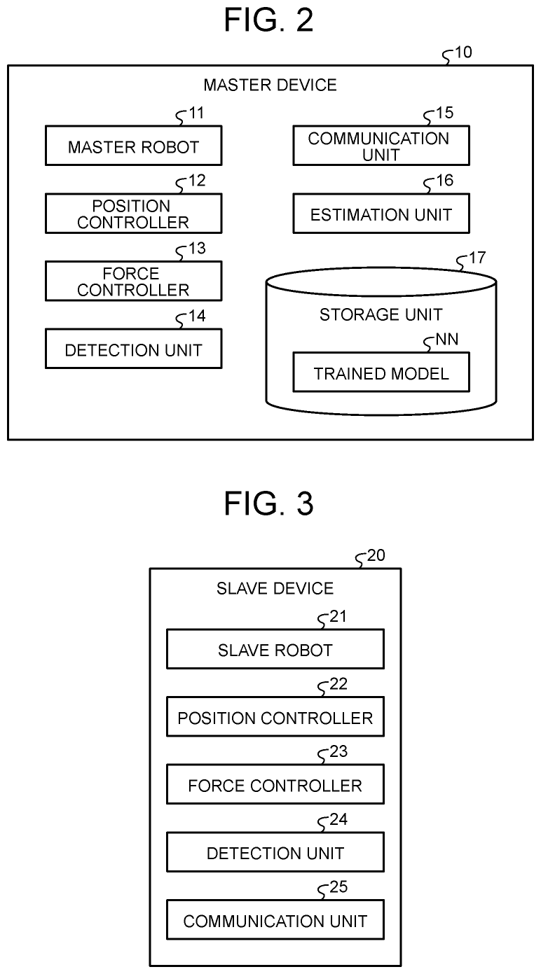

The master device 10 and the slave device 20 will be described with reference to FIGS. 2 and 3 as well.

FIG. 2 is a diagram illustrating an example of a schematic configuration of a master device. The master device 10 includes, in addition to the master robot 11 described above, a position controller 12, a force controller 13, a detection unit 14, a communication unit 15, an estimation unit 16, and a storage unit 17.

While details of the position controller 12 and the force controller 13 will be described below, only the force controller 13 is used in the unilateral control, and both the position controller 12 and the force controller 13 are used in the bilateral control.

The detection unit 14 detects the state of the master robot 11. An example of the detected state is an external force (for example, at the distal end) of the master robot 11, and this force is acquired from the above-described force sensor or the like. Other examples of the state to be detected include a torque reference value, an angle (joint angle), and an angular velocity (joint angular velocity) of the joint. The torque reference value, which substantially corresponds to a current value input to the master robot 11, is naturally sensed by the master device 10, and thus is detectable. The joint angle is acquired from an encoder in an actuator (not illustrated) provided at the joint of the master robot 11, for example. The joint angular velocity is obtained as a derivative of the joint angle with respect to time. Still other examples of the detected state include an acceleration reference value, the position, the speed, and the like of the distal end, which are input to the master robot 11. The acceleration reference value, which is a value as a basis of the above-described torque reference value, is naturally sensed by the master device 10 similarly to the torque reference value, and thus, is detectable. The position and speed of the distal end are specified from the above-described information such as a joint angle and joint angular velocity, for example, and thus are detectable.

The communication unit 15 communicates with other devices. An example of other devices is the slave device 20. For example, the communication unit 15 receives information to be used by the master device 10 from the slave device 20 via the network 5. Furthermore, the communication unit 15 transmits information acquired by the master device 10 to the slave device 20 via the network 5.

Here, the slave device 20 will be described first, and then the master device 10 will be described.

FIG. 3 is a diagram illustrating an example of a schematic configuration of the slave device. The slave device 20 includes, in addition to the slave robot 21 described above, a position controller 22, a force controller 23, a detection unit 24, and a communication unit 25.

While details of the position controller 22 and the force controller 23 will be described below, only the position controller 22 is used in the unilateral control, and both the position controller 22 and the force controller 23 are used in the bilateral control.

The detection unit 24 detects the state of the slave robot 21. An example of the detected state is an external force of the slave robot 21 (for example, the distal end 21E), and this is acquired from the above-described force sensor or the like. Other examples of the state to be detected include a torque reference value, an angle (joint angle), and an angular velocity (joint angular velocity) of the joint 21J. The torque reference value, which substantially corresponds to a current value input to the slave robot 21, is naturally sensed by the slave device 20, and thus is detectable. The joint angle is acquired from an encoder in an actuator (not illustrated) provided at the joint 21J of the slave robot 21, for example. The joint angular velocity is obtained as a derivative of the joint angle with respect to time. Still other examples of the detected state include an acceleration reference value, the position, the speed, and the like of the distal end 21E, which are input to the slave robot 21. The acceleration reference value, which is a value as a basis of the above-described torque reference value, is naturally sensed by the slave device 20 similarly to the torque reference value, and thus, is detectable. The position of the distal end 21E and the speed of the distal end 21E are specified from the above-described information such as a joint angle and joint angular velocity, for example, and thus are detectable.

The communication unit 25 communicates with other devices. An example of other devices is the master device 10. For example, the communication unit 25 receives information to be used by the slave device 20 from the master device 10 via the network 5. Furthermore, the communication unit 25 transmits the information acquired by the slave device 20 to the master device 10 via the network 5.

The estimation unit 16 and the storage unit 17 of the master device 10 will be described with reference back to FIG. 2.

The estimation unit 16 estimates the future state information of the slave robot 21 based on the state information of the master robot 11 and the slave robot 21. The state information includes various information for specifying the state of the master robot 11 or the slave robot 21. Examples of the state information include the position (distal end, for example) of the master robot 11 and the position and speed of the slave robot 21 (specifically, the distal end 21E, for example) acquired by the master device 10. The state information may be, for example, time-series information (that is, information up to the present) indicating the states of the master robot 11 and the slave robot 21 for at each time.

The future state information is state information of the slave robot 21 at a time after a lapse of a predetermined period from a time corresponding to the state information of the slave robot 21 acquired by the master device 10. The predetermined period may be determined based on a communication delay time between the master device 10 and the slave device 20. More specifically, the predetermined period may be determined based on a round-trip communication delay time between the master device 10 and the slave device 20. The communication delay time may be determined based on data such as design data and experimental data of the control system 1. The communication delay time will be described below with reference to FIGS. 5 to 7 as well. Details of the estimation by the estimation unit 16 will be described below.

With reference back to FIG. 3, the following will describe the controllers included in the control system 1, more specifically, the position controller 12 and the force controller 13 of the master device 10 and the position controller 22 and the force controller 23 of the slave device 20. In the following description, the position controller 12 and the position controller 22 may be collectively referred to as a “position controller 12 and the like”. The force controller 13 and the force controller 23 may be collectively referred to as a “force controller 13 and the like”.

The position controller 12 and the like controls the master robot 11 and the slave robot 21 so as to achieve the correspondence between the position of the master robot 11 and the position of the slave robot 21. The correspondence between positions here means that the positions of the corresponding parts of the master robot 11 and the slave robot 21 have a correspondence relationship. For example, the master robot 11 and the slave robot 21 are controlled so as to allow the position of the distal end of the master robot 11 and the position of the distal end 21E of the slave robot 21 to have a correspondence relationship.

Specifically, the position controller 12 controls the position of the master robot 11 in accordance with a position command value designated by the user. An example of the control position is the position of the distal end of the master robot 11 (arm end position). This control is performed in accordance with information designating the position of the distal end (arm end position command). The arm end position command is generated in accordance with a user' operation or generated by the position controller 12. The position controller 12 controls the rotation (rotation speed, rotation angular velocity, torque, etc.) of the joint so as to allow the distal end of the master robot 11 to be located at a position according to the arm end position command.

The position controller 22 controls the position of the slave robot 21 so as to follow the position command value designated by the user. An example of the control position is a position (arm end position) of the distal end 21E of the slave robot 21. This control is performed in accordance with the arm end position command. The position controller 22 controls, for example, the rotation (rotation speed, rotation angular velocity, torque, etc.) of the joint 21J so as to allow the distal end 21E of the slave robot 21 to be positioned in accordance with the arm end position command.

The force controller 13 and the like controls the master robot 11 and the slave robot 21 so as to achieve the correspondence between the external force of the master robot 11 and the external force of the slave robot 21. The external force between positions here means that the external forces of the corresponding parts of the master robot 11 and the slave robot 21 have a correspondence relationship. For example, the master robot 11 and the slave robot 21 are controlled so as to allow the external force of the distal end of the master robot 11 and the external force of the distal end 21E of the slave robot 21 to have a correspondence relationship.

The position control and the force control by the position controller 12 and the like and the force controller 13 and the like are performed based on the estimation result obtained by the estimation unit 16. Details will be described below.

The storage unit 17 of the master device 10 will be described. The storage unit 17 stores various types of information needed for processing executed in the master device 10. The information stored in the storage unit 17 include a trained model NN as illustrated in FIG. 2.

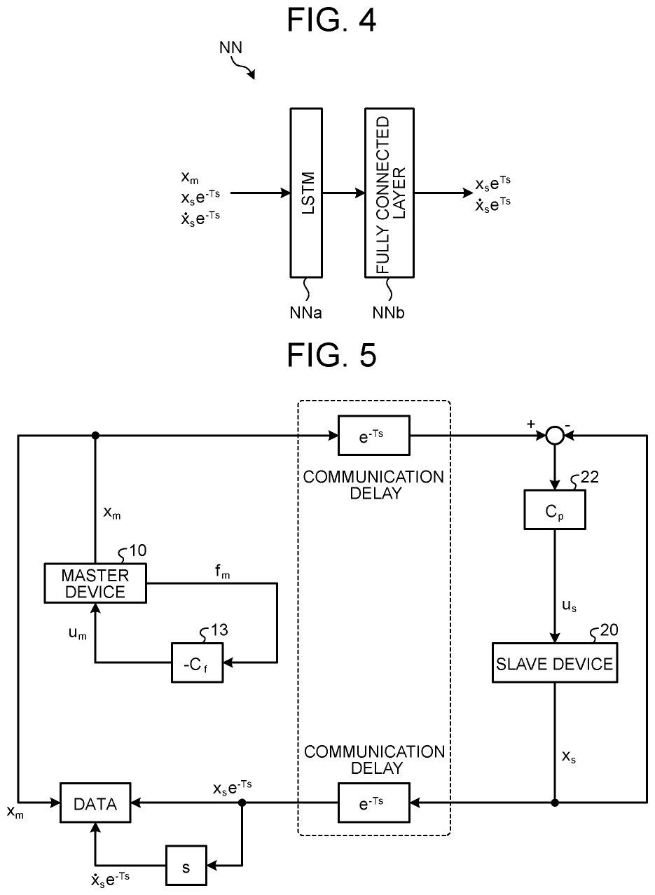

The trained model NN estimates the future state information of the slave robot 21 of the slave device 20. The trained model NN will be described with reference to FIGS. 4 and 5 as well.

FIG. 4 is a diagram illustrating an example of a schematic configuration of a trained model. In this example, the trained model NN is a deep neural network, and includes a long short-term memory (LSTM) block NNa and a fully connected layer NNb.

Using training data (such as labeled training data), the trained model NN is generated such that, after having an input of state information of the master robot 11 and the slave robot 21, the model NN will output future state information of the slave robot 21. In this example, the state information input to the trained model NN includes a position (xₘ) of the distal end of the master robot 11, a position (xₛe^{-Ts}) and a speed (xₛe^{-Ts} dot) of the distal end 21E of the slave robot 21. Note that the position (xₛe^{-Ts}) and the speed (xₛe^{-Ts} dot) of the distal end 21E here are information acquired by the master device 10, and are thus information (past information) delayed by the communication delay time (T) between the master device 10 and the slave device 20. Also note that the future state information of the slave robot 21 output by the trained model NN is a future position (xₛe^{Ts}) and a future speed (xₛe^{Ts} dot) of the distal end 21E of the slave robot 21, and thus is information shifted to the future by the communication delay time (T). Incidentally, the communication delay time (T) is a delay time occurring in communication from the master device 10 to the slave device 20 or communication from the slave device 20 to the master device 10. The round-trip communication delay time between the master device 10 and the slave device 20 is the sum of the individual communication delay times (for example, 2T).

As described above, the state information may be time-series information (time-series data). Such state information is suitable to be input to the trained model NN using an LSTM block NNa suitable for time-series data. For example, the estimation accuracy of the future state information can be improved as compared with the case of using a multilayer perceptron. The trained model NN can also be considered as a model (system) that predicts a future response by a communication delay from time-series data up to the current time.

FIG. 5 is a diagram schematically illustrating an example of acquisition of training data. Unless otherwise specified in the drawing, the illustrated processing is executed by the force controller 13 of the master device 10 and the position controller 22 of the slave device 20, for example. For convenience of description, FIG. 5 illustrates the master device 10 separately from the force controller 13 included in the master device 10. The slave device 20 is illustrated separately from the position controller 22 included in the slave device 20.

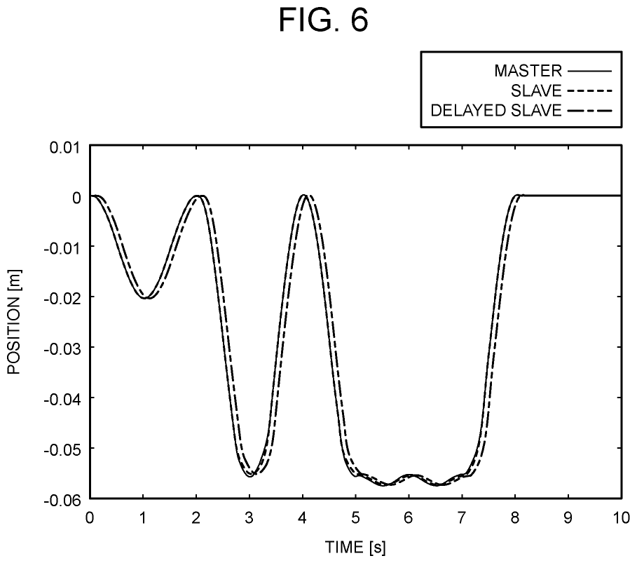

A unilateral control system from the master device 10 to the slave device 20 is configured. The left part of FIG. 6 corresponds to a system on the master device 10 side. The right part of FIG. 6 corresponds to a system on the side of the slave device 20. Transmission and reception (communication) of information between the master device 10 and the slave device 20 is performed via the network 5 described above with reference to FIG. 1.

The system constructed on the side of the master device 10 is a force control system that follows 0 N. The force controller 13 calculates an input (uₘ) to the master robot 11 so as to bring the external force (fₘ) of the distal end of the master robot 11 of the master device 10 closer to 0. Examples of the input (uₘ) include an acceleration reference value and a torque reference value. In this example, the force controller 13 calculates the input (uₘ) to the master robot 11 by multiplying an external force (fₘ) at the distal end of the master robot 11 by −C_{f}. The position (xₘ) of the distal end of the master robot 11 during such control is transmitted to the slave device 20 with a delay of the communication delay time (T).

The system constructed on the side of the slave device 20 is a position control system that follows the position of the distal end of the master robot 11. The position controller 22 calculates an input (uₛ) to the slave robot 21 so as to bring the position (xₛ) of the distal end 21E of the slave robot 21 of the slave device 20 closer to the position of the distal end of the master robot 11. In this example, the position controller 22 calculates the input (uₛ) to the slave robot 21 by multiplying a value obtained by subtracting the position (xₛ) of the distal end 21E of the slave robot 21 from the position of the distal end of the master robot 11 by Cₚ. The position (xₛ) of the distal end 21E of the slave robot 21 under such control is transmitted (as feedback) to the master device 10 with a delay (xₛe^{-Ts}) by the communication delay time (T).

The master device 10 side acquires, as data, the position (xₘ) of the distal end of the master robot 11, the position (xₛe^{-Ts}) and the speed (xₛe^{-Ts} dot) of the distal end 21E of the slave robot 21. By acquiring the data in time series, the position (xₛe^{Ts}) and the speed (xₛe^{Ts} dot) of the distal end 21E of the future slave robot 21 are also acquired. The data obtained in this manner, specifically, the position (xₘ) of the distal end of the master robot 11, the position (xₛe^{-Ts}) and speed (xₛe^{-Ts} dot) of the distal end 21E of the slave robot 21, and the future position (xₛe^{Ts}) and future speed (xₛe^{Ts} dot) of the distal end 21E of the slave robot 21, are acquired as inputs and outputs of training data for the trained model NN. The training data is acquired by performing all sorts of unilateral control within a range of positions and speeds of the distal ends of the master robot 11 and the slave robot 21 assumed at the time of bilateral control described below. At this time, the master robot 11 is operated to set the slave robot 21 to be always in a no-load state (non-contact state).

FIG. 6 is a diagram schematically illustrating an example of training data. The horizontal axis of the graph indicates time (s), and the vertical axis indicates position (m). The graph line noted as master and the graph line noted as slave indicate current responses of the master robot 11 and the slave robot 21, which correspond to inputs. The graph line noted as delayed slave indicates the future response of the slave robot 21, which corresponds to labeled training data.

For example, the training data of the trained model NN is acquired as described above. The trained model NN is generated using the acquired training data. The generation (learning) of the trained model NN may be performed outside the control system 1 (an information processing device or the like (not illustrated)). The generated trained model NN is stored in the storage unit 17 of the master device 10.

Returning to FIG. 2. The estimation unit 16 of the master device 10 estimates the future position (xₛe^{Ts}) and speed (xₛe^{Ts} dot) of the distal end 21E of the slave robot 21 by using the position (xₘ) of the distal end of the master robot 11 acquired by the master device 10, the position (xₛe^{-Ts}) and speed (xₛe^{-Ts} dot) of the distal end 21E of the slave robot 21, and the above-described trained model NN. The estimation result is used for position control by the position controller 12 and the like and force control by the force controller 13 and the like at the time of bilateral control described below.

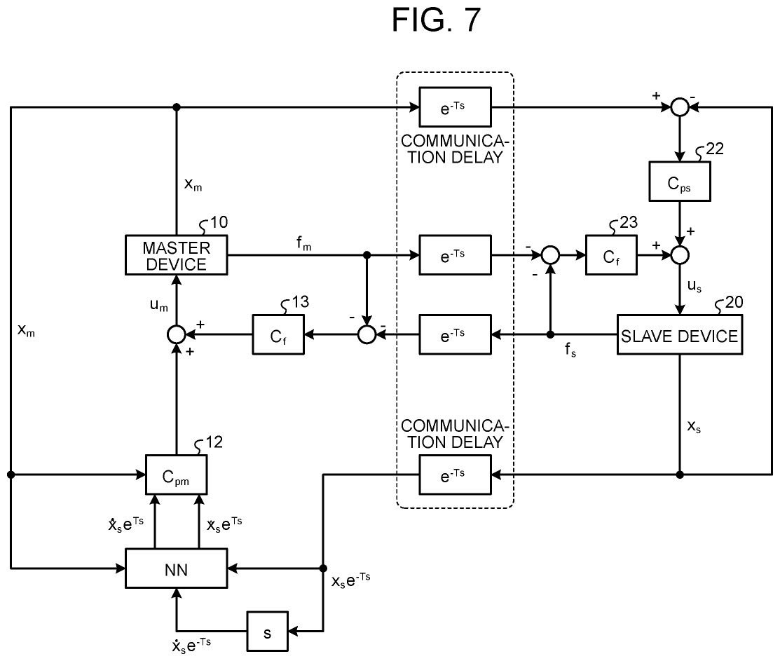

A bilateral control system constructed by the control system 1 (FIG. 1) will be described. In the bilateral control system, the positions and the external forces of the master robot 11 and the slave robot 21 are transmitted to each other such that the positions and the external forces correspond to each other. This will be described with reference to FIG. 7.

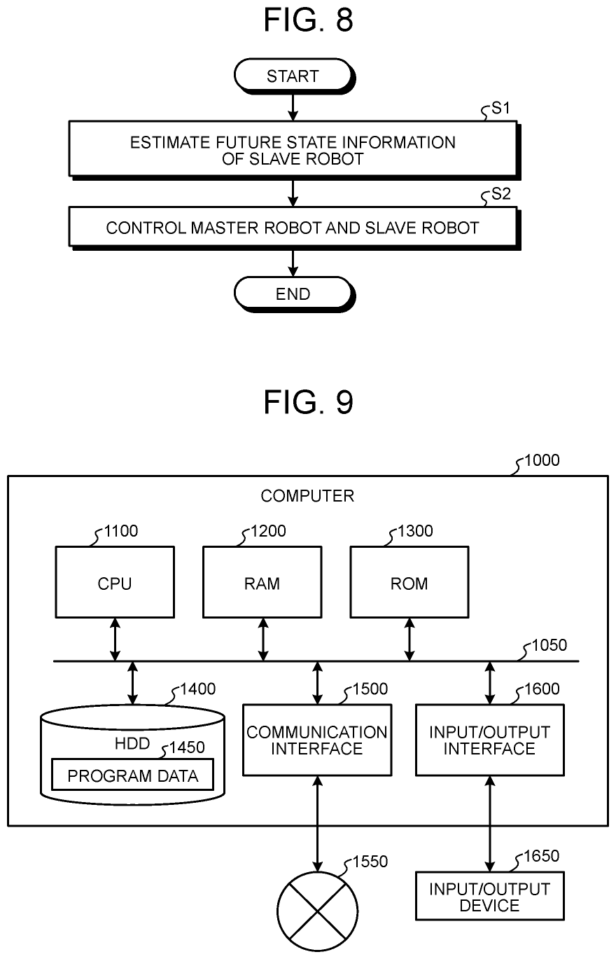

FIG. 7 is a diagram schematically illustrating an example of bilateral control. The left part of FIG. 8 corresponds to a system on the master device 10 side. The right part of FIG. 7 corresponds to a system on the slave device 20 side. Transmission and reception (communication) of information between the master device 10 and the slave device 20 is performed via the network 5 described above with reference to FIG. 1. Unless otherwise specified, the processes in the diagram are performed individually by the position controller 12 and the force controller 13 on the master device 10, and by the position controller 22 and the force controller 23 on the slave device 20 (calculated by the local controller).

The following control targets are given between the position (xₘ) and the external force (fₘ) of the distal end of the master robot 11 and the position (xₛ) and the external force (fₛ) of the distal end 21E of the slave robot 21.

xₘ−xₛ=0 (1)

fₘ−fₛ=0 (2)

The above Formula (1) expresses matching between the position (xₘ) of the distal end of the master robot 11 and the position (xₛ) of the distal end 21E of the slave robot 21. Formula (2) expresses the law of action and reaction between the external force (fₘ) at the distal end of the master robot 11 and the external force (fₛ) at the distal end 21E of the slave robot 21. The external force (fₛ) and the external force (fₘ) are detected by the force sensor (the force sensor 21F or the like) as described above with reference to FIG. 1.

On the master device 10 side, both the external force (fₘ) of the distal end of the master robot 11 and the external force (fₛe^{-Ts}) of the distal end 21E of the slave robot 21 are subtracted. The force controller 13 multiplies the value by C_{f} and outputs the result. The output of the force controller 13 is added to the input (uₘ) from the position controller 12 to the master robot 11.

On the side of the slave device 20, the position (xₛ) of the distal end 21E of the slave robot 21 is subtracted from the position (xₘe^{-Ts}) of the distal end of the master robot 11. The position controller 22 multiplies the value by Cₚₛ and outputs the result. A value obtained by adding the output from the force controller 23 to the output from the position controller 22 is to be an input (uₛ) to the slave robot 21 of the slave device 20. Both the external force (fₛ) of the distal end 21E of the slave robot 21 and the external force (fₘe^{-Ts}) of the distal end of the master robot 11 are subtracted. The force controller 23 multiplies the value by C_{f} and outputs the result. The output of the force controller 23 is added to the input (uₛ) from the position controller 22 to the slave robot 21.

In the master device 10, the position (xₘ) of the distal end of the master robot 11, the position (xₛe^{-Ts}) of the distal end 21E of the slave robot 21, and the speed (xₛe^{-Ts} dot) obtained by differentiating (S) the position are input to the trained model NN. Specifically, the estimation unit 16 inputs the position (xₘ) of the distal end of the master robot 11, the position (xₛe^{-Ts}) of the distal end 21E, and the speed (xₛe^{-Ts} dot) to the trained model NN.

The trained model NN outputs a future position (xₛe^{Ts} hat) and a future speed (xₛe^{Ts} hat and dot) of the distal end 21E of the slave robot 21. These values are acquired by the estimation unit 16.

The position controller 12 calculates an input (uₘ) to the master robot 11 based on the position (xₘ) of the distal end of the master robot 11, the future position (xₛe^{Ts} hat) of the distal end 21E, and the future speed (xₛe^{Ts} hat and dot) of the distal end 21E.

In an assumable case of bilateral control constructed based on the control target without estimating the future position (xₛe^{Ts} hat) and the speed (xₛe^{Ts} hat and dot) using the trained model NN under the condition where there is a communication delay as illustrated in FIG. 7, the results would be obtained as in the following Formulas.

uₘ=−C_{f}(fₘ+fₛe^{-Ts})+Cₚₘ(xₘ−xₛe^{-Ts}) (3)

uₛ=−C_{f}(fₘe^{-Ts}+fₛ)+Cₚₛ(xₘe^{-Ts}−xₛ) (4)

According to the above Formulas (3) and (4), the position (xₘ) of the distal end of the master robot 11 and the position (xₛe^{-Ts}) of the distal end 21E of the slave robot 21 are not synchronized at a same time. This would lead to significant deterioration of the operability in the non-contact state as well as deterioration of the stability of the control system, resulting in instability of the control system particularly at the time of contact with an external environment.

In this regard, as described above, the master device 10 side estimates (predicts) the future response (position (xₛe^{Ts})) of the distal end 21E of the slave robot 21 in real time using (implementing) the trained model NN. The position control system on the master device 10 side is constructed based on the estimated value and the current response (position (xₘ)) of the master robot 11. In a case where the trained model NN accurately outputs the future prediction value (xₛ hat), the above Formulas (3) and (4) are overwritten as follows.

uₘ=−C_{f}(fₘ+fₛe^{-Ts})+Cₚₘ(xₘ−\hat{x}ₛ) (5)

uₛ=−C_{f}(fₛe^{-Ts}+fₛ)−Cₚₛ(xₘe^{-Ts}−xₛ) (6)

The master device 10 side can virtually assume that there is no communication delay in the position and speed of the distal end 21E of the slave robot 21. Therefore, the position of the distal end of the master robot 11 and the position of the distal end 21E of the slave robot 21 are synchronized with each other at a same time. As a result, it is possible to suppress deterioration in operability and stability.

According to the control system 1 described above, the master device 10 side has a control system constructed using future state information of the slave robot 21 (the future position and future speed of the distal end 21E), making it possible to design with assumption of no communication delay. This can achieve both improvement of operability and stability at the time of contact, which have been difficult with conventional technologies.

For example, in contrast to the cases using a delay compensator such as the Smith predictor, with the technique according to the present embodiment, the above-described effect can be obtained without using such an additional delay compensator.

The master device 10 side can handle situations assuming there is no delay in the position of the distal end 21E and the speed of the distal end 21E of the slave robot 21. Therefore, an existing controller can be used without considering the communication delay without a need to design a complicated communication delay controller such as a method based on a passivity theory.

FIG. 8 is a flowchart illustrating an example of processing (control method) executed in the control system. Portions overlapping with the description above will not be described again.

Step S1 estimates the future state information of the slave robot. As described above with reference to FIG. 7, the estimation unit 16 of the master device 10 estimates the future position (xₛe^{Ts}) and speed (xₛe^{Ts} dot) of the distal end 21E of the slave robot 21 using the trained model NN.

In step S2, the master robot and the slave robot are controlled. As described above with reference to FIG. 7, based on the estimation result in step S1 described above, the position controller 12 and the like and the force controller 13 and the like control the master robot 11 and the slave robot 21 such that the position (xₘ) and the external force (fₘ) of the master robot 11 correspond to the position (xₛe^{Ts}) and the external force (fₛe^{Ts}) of the slave robot 21, respectively.

Bilateral control is performed by repeatedly executing the above processing, for example.

### 2. Modification

In the bilateral control, scaling may be performed on an external force and a position. For example, in a case where scaling is performed on the position and force of the distal end 21E of the slave robot 21, control on the master device 10 side is expressed as follows.

uₘ=−C_{f}(fₘ+αfₛe^{-Ts})+Cₚₘ(xₘ−β\hat{x}ₛ) (7)

The control on the side of the slave device 20 is expressed as follows, for example.

uₛ=−C_{f}(fₘe^{-Ts}+αfₛ)−Cₚₛ(xₘe^{-Ts}−βxₛ) (8)

Here, α (>0) is a scaling factor of the external force. β (>0) is a scaling factor of the position.

By performing scaling as described above, it is possible to facilitate remote bilateral control in surgery requiring fine work, for example. Since the bilateral control is as described above with reference to FIG. 7, redundant description will not be given.

In the above embodiment, the medical bilateral control system for surgery or the like has been described as an example of the application of the control system 1. However, the control system 1 may be used for various other applications. Examples of other applications includes a bilateral control robot for extreme environments. The slave robot can be sent to an environment not easily accessible by humans, such as a disaster site or outer space, enabling bilateral operation from a remote location with excellent operability and stability. Another example of other applications is a network-based control system. By predicting a future response for a robot connected to a network in a factory or the like, it is possible to design a controller that is more robust than a Smith predictor and easier than the passivity theory.

The mechanism included in the robot is not limited to the arm. The robot may have various mechanisms depending on the application.

The position control object, the position/speed estimation object, and the like are not limited to the distal end of the robot. Any part of the robot may be a position control object and a position/speed estimation object.

The location of each function of the control system 1 may be provided in any device of the master device 10 and the slave device 20. This is because these devices can communicate with each other. Some functions may be provided in a device outside the control system 1 (for example, an external server).

### 3. Hardware Configuration Example

FIG. 9 is a diagram illustrating an example of a hardware configuration of the device. The device such as the master device 10 is implemented by including a computer 1000 having a configuration as illustrated in FIG. 9, for example.

The computer 1000 includes a CPU 1100, RAM 1200, read only memory (ROM) 1300, a hard disk drive (HDD) 1400, a communication interface 1500, and an input/output interface 1600. Individual components of the computer 1000 are interconnected by a bus 1050.

The CPU 1100 operates based on a program stored in the ROM 1300 or the HDD 1400 so as to control each of components. For example, the CPU 1100 develops the program stored in the ROM 1300 or the HDD 1400 into the RAM 1200 and executes processing corresponding to various programs.

The ROM 1300 stores a boot program such as a basic input output system (BIOS) executed by the CPU 1100 when the computer 1000 starts up, a program dependent on hardware of the computer 1000, or the like.

The HDD 1400 is a non-transitory computer-readable recording medium that records a program executed by the CPU 1100, data used by the program, or the like. Specifically, the HDD 1400 is a recording medium that records a program that executes control processing according to the present disclosure, which is an example of program data 1450.

The communication interface 1500 is an interface for connecting the computer 1000 to an external network 1550 (for example, the Internet). For example, the CPU 1100 receives data from other devices or transmits data generated by the CPU 1100 to other devices via the communication interface 1500.

The input/output interface 1600 is an interface for connecting an input/output device 1650 with the computer 1000. For example, the CPU 1100 receives data from an input device such as a keyboard or a mouse via the input/output interface 1600. In addition, the CPU 1100 transmits data to an output device such as a display, a speaker, or a printer via the input/output interface 1600. Furthermore, the input/output interface 1600 may function as a media interface for reading a program or the like recorded on predetermined recording medium (or simply medium). Examples of the media include optical recording media such as a digital versatile disc (DVD) or a phase change rewritable disk (PD), a magneto-optical recording medium such as a magneto-optical disk (MO), a tape medium, a magnetic recording medium, and semiconductor memory.

For example, when the computer 1000 functions as the master device 10 according to the embodiment, the CPU 1100 of the computer 1000 executes the program loaded on the RAM 1200 so as to implement the functions of the master device 10 or the like. In addition, the HDD 1400 stores a program that executes information processing according to the present disclosure (estimation processing or the like by the estimation unit 16) and data in the storage unit 17. While the CPU 1100 executes program data 1450 read from the HDD 1400, the CPU 1100 may acquire these programs from another device via the external network 1550, as another example.

### 4. Effects

The control system 1 described above is specified as follows, for example. As described with reference to FIGS. 1 to 3 and the like, the control system 1 includes the estimation unit 16 and the controllers (the position controller 12, the force controller 13, the position controller 22, and the force controller 23). Using the trained model NN and state information of the master robot 11 and the slave robot 21 acquired by the master device 10 among the master device 10 including the master robot 11 and the slave device 20 including the slave robot 21 (for example, an arm), the estimation unit 16 estimates future state information of the slave robot 21 (for example, the distal end 21E) at a time after a predetermined period from a time corresponding to the state information. Based on the estimation result obtained by the estimation unit 16, the controller controls the master robot 11 and the slave robot 21 such that the position and the external force of the master robot 11 correspond to the position and the external force of the slave robot 21. After having input of the state information, the trained model NN outputs the future state information. The predetermined period may be a period defined based on a communication delay time (for example, a round-trip communication delay time) between the master device 10 and the slave device 20.

The control system 1 described above performs bilateral control in which the positions and external forces of the master robot 11 and the slave robot 21 are set to correspond to each other based on the estimation result of the future state information of the slave robot 21. By using the future state information of the slave robot 21 in this manner, it is possible to reduce the influence of the communication delay time between the master device 10 and the slave device 20, leading to improvement of the operability of the bilateral control.

As described with reference to FIG. 4 and the like, the trained model NN may include the LSTM block NNa, and the state information may be time-series information. With this configuration, the estimation accuracy of the future state information can be improved as compared with the case of using the multilayer perceptron, for example.

As described with reference to FIG. 7 and the like, the state information may include the position (xₘ) of the master robot 11, the position (xₛe^{-Ts}) and speed (xₛe^{-Ts} dot) of the slave robot 21, and the future state information may include the position (xₛe^{-Ts} hat) and speed (xₛe^{-Ts} hat and dot) of the slave robot 21. For example, the future state information of the slave robot 21 can be estimated based on such state information of the master robot 11 and the slave robot 21.

The controller may control the master robot 11 and the slave robot 21 such that the position and the external force of the master robot 11 correspond to the scaled position (for example, the scaling factor is β) and the external force (for example, the scaling factor is α) of the slave robot 21, respectively. This makes it possible to facilitate remote bilateral control in surgery requiring fine work.

The control method described with reference to FIG. 8 and the like is also an aspect of the present disclosure. The control method includes: estimating, by the control system 1 using the trained model NN and state information of the master robot 11 and the slave robot 21 acquired by the master device 10 among the master device 10 including the master robot 11 and the slave device 20 including the slave robot 21, future state information of the slave robot 21 at a time after a predetermined period from a time corresponding to the state information (step S1); and controlling, by the control system 1, the master robot 11 and the slave robot 21 based on an estimation result so as to achieve a correspondence of a position and an external force of the master robot 11, and a position and an external force of the slave robot 21, respectively (step S2). By such a control method, as described above, the operability of the bilateral control can be improved as well.

Note that the effects described in the present disclosure are merely examples and are not limited to the disclosed contents. There may be other effects.

The embodiments of the present disclosure have been described above. However, the technical scope of the present disclosure is not limited to the above-described embodiments, and various modifications can be made without departing from the scope of the present disclosure. Moreover, it is allowable to combine the components across different embodiments and modifications as appropriate.

Note that the present technique can also have the following configurations.
(1) A control system comprising:
   an estimation unit that estimates future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and
   a controller that controls the master robot and the slave robot based on an estimation result obtained by the estimation unit so as to achieve a correspondence between a position and an external force of the master robot and a position and an external force of the slave robot, respectively,
   wherein the trained model works such that, after having input of the state information, the trained model outputs the future state information.
(2) The control system according to (1),
   wherein the predetermined period is a period defined based on a communication delay time between the master device and the slave device.
(3) The control system according to (1) or (2),
   wherein the predetermined period is a period defined based on a round-trip communication delay time between the master device and the slave device.
(4). The control system according to any one of (1) to (3),
wherein the trained model includes a long short-term memory (LSTM) block, and
   the state information is time-series information.
(5). The control system according to any one of (1) to (4),
   wherein the state information includes the position of the master robot, and the position and a speed of the slave robot, and
   the future state information includes the position and the speed of the slave robot.
(6). The control system according to any one of (1) to (5),
   wherein the state information includes a position of a distal end of the master robot, and a position and a speed of a distal end of the slave robot, and
   the future state information includes the position and the speed of the distal end of the slave robot.
(7). The control system according to any one of (1) to (6),
   wherein the slave robot includes an arm.
(8) The control system according to any one of (1) to (7),
   wherein the controller controls the master robot and the slave robot so as to achieve a correspondence between the position and the external force of the master robot and a position and an external force of the slave robot which has been scaled, respectively.
9. A control method comprising:
   estimating, by a control system, future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and
   controlling, by the control system, the master robot and the slave robot based on an estimation result so as to achieve a correspondence between a position and an external force of the master robot and a position and an external force of the slave robot, respectively.

### REFERENCE SIGNS LIST

1 CONTROL SYSTEM
10 MASTER DEVICE
11 MASTER ROBOT
12 POSITION CONTROLLER
13 FORCE CONTROLLER
14 DETECTION UNIT
15 COMMUNICATION UNIT
16 ESTIMATION UNIT
17 STORAGE UNIT
20 SLAVE DEVICE
21 SLAVE ROBOT
22 POSITION CONTROLLER
23 FORCE CONTROLLER
24 DETECTION UNIT
25 COMMUNICATION UNIT
NN TRAINED MODEL
NNa LSTM BLOCK
NNb FULLY CONNECTED LAYER

1. A control system comprising:
an estimation unit that estimates future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and
a controller that controls the master robot and the slave robot based on an estimation result obtained by the estimation unit so as to achieve a correspondence between a position and an external force of the master robot and a position and an external force of the slave robot, respectively,
wherein the trained model works such that, after having input of the state information, the trained model outputs the future state information.

2. The control system according to claim 1,
wherein the predetermined period is a period defined based on a communication delay time between the master device and the slave device.

3. The control system according to claim 1,
wherein the predetermined period is a period defined based on a round-trip communication delay time between the master device and the slave device.

4. The control system according to claim 1,
wherein the trained model includes a long short-term memory (LSTM) block, and
the state information is time-series information.

5. The control system according to claim 1,
wherein the state information includes the position of the master robot, and the position and a speed of the slave robot, and
the future state information includes the position and the speed of the slave robot.

6. The control system according to claim 1,
wherein the state information includes a position of a distal end of the master robot, and a position and a speed of a distal end of the slave robot, and
the future state information includes the position and the speed of the distal end of the slave robot.

7. The control system according to claim 1,
wherein the slave robot includes an arm.

8. The control system according to claim 1,
wherein the controller controls the master robot and the slave robot so as to achieve a correspondence between the position and the external force of the master robot and a position and an external force of the slave robot which has been scaled, respectively.

9. A control method comprising:
estimating, by a control system, future state information of a slave robot at a time after a predetermined period from a time corresponding to state information of a master robot and the slave robot, the state information being acquired by a master device among the master device and a slave device, the master device including the master robot while the slave device including the slave robot, the estimation of the future state information being performed by using the state information of the master robot and the slave robot and using a trained model; and
controlling, by the control system, the master robot and the slave robot based on an estimation result so as to achieve a correspondence between a position and an external force of the master robot and a position and an external force of the slave robot, respectively.
